(19) United States
(12) Patent Application Publication
(10) Pub. No.: US 2003/0130499 A1
(43) Pub. Date: Jul. 10, 2003

(54) Isolation of nucleic acids

(76) Inventor: Matthew John Baker, Maidstone (GB)

Correspondence Address: DANN DORFMAN HERRELL & SKILLMAN, SUITE 720, 1601 MARKET STREET, PHILADELPHIA, PA, 19103-2307, US

(21) Appl. No.: 10/300,990

(22) Filed: Nov. 21, 2002

Related U.S. Application Data
(63) Continuation of application No. 09/586,009, filed on Jun. 2, 2000.

(30) Foreign Application Priority Data
Dec. 4, 1998 (WO) PCT/GB98/03602
Dec. 6, 1997 (GB) 9725839.6
Jul. 17, 1998 (GB) 9815541.9

(51) Int. Cl. C07H 21/04; C12N 1/08

(52) U.S. Cl. 536/25.4; 435/270

(57) ABSTRACT

Disclosed is a method of extracting nucleic acids from blood which comprises contacting blood cells, preferably after lysing with an activated solid phase at one pH to immobilize the nucleic acids and then removing the nucleic acids at a higher pH when the charge has been reversed or neutralized. The solid phase can be beads activated by a histidine as a binding agent. The beads can be fluidized by sucking the blood with air up through a column containing the beads to improve contact and prevent clogging.

The present invention relates to a method for extracting nucleic acids and other biomolecules from biological material, particularly blood.

There is a very large demand for DNA analysis for a range of purposes and this has lead to the requirement for quick, safe, high throughput methods for the isolation and purification of DNA and other nucleic acids.

Samples for use for DNA identification or analysis can be taken from a wide range of sources such as biological material such as animal and plant cells, faeces, tissue etc. also samples can be taken from soil, foodstuffs, water etc.

Existing methods for the extraction of DNA include the use of phenol/chloroform, salting out, the use of chaotropic salts and silica resins, the use of affinity resins, ion exchange chromatography and the use of magnetic beads. Methods are described in U.S. Pat. Nos. 5,057,426, 4,923,978, EP Patents 0512767 A1 and EP0515484B and WO 95/13368, WO 97/10331 and WO 96/18731. These patents and patent applications disclose methods of adsorbing nucleic acids on to a solid support and then isolating the nucleic acids. The previously used methods use some type of solvent to isolate the nucleic acids and these solvents are flammable, combustible or toxic.

Blood is one of the most abundant sample sources for DNA analysis as blood samples are routinely taken for a wide range of reasons. However because of the viscous and proteinaceous nature of blood using known DNA extraction methods it has proved difficult to accomplish using automation due to the difficulties of handling large volumes containing relatively small amounts of DNA. Hitherto nucleic acid extraction has been partially automated only by using hazardous reagents and slow processing times.

I have now devised an improved method for the extraction of nucleic acids and other biomolecules from blood and other biological materials.

According to the invention there is provided a method for the extraction of biomolecules from biological material which method comprises contacting the biological material with a solid phase which is able to bind the biomolecules to it at a first pH and then extracting the biomolecules bound to the solid phase by elution using an elution solvent at a second pH.

The method is particularly useful if the biological material is blood, but the method can be used for a range of applications substances such as Plasmid and vector isolation and plant DNA extraction.

Preferably the cells in the blood are lysed to release nucleic acids and known lysing agents and methods can be used, such as contacting with ionic and non ionic detergents, hypotonic solutions of salts, proteases, chaotropic agents, solvents, using pH changes or heat. A method of lysing cells to isolate nucleic acid is described in WO 96/00228.

When the biological material consists of blood the samples can optionally be diluted with water or other diluent in order to make it easier to manipulate and to process.

Dilutions up to ten times can be used and in general more dilution can be better and it is a feature of the present invention that it allows low dilution of blood to be possible.

The solid phase with which the blood is contacted, can be a formed of a material which has a natural affinity for nucleic acids or it can be formed of a material which has its surface treated with an agent which will cause nucleic acids to bind to it or increase its affinity for nucleic acids. Suitable materials include controlled pore glass, polysaccharide (agarose or cellulose), other types of silica/glass, ceramic materials, porous plastic materials such as porous plastic plugs which in a single moulded part or as an insert in a standard tube, polystyrene beads para magnetic beads etc. The size and porosity is not critical and can vary and be selected for particular applications.

Suitable means for treating the surface of the solid phase or for derivitising it include treating it with a substance which can introduce a charge e.g. a positive charge on the surface or a hydrophilic or hydrophobic surface on the solid phase e.g. hydroxyl groups, nitrate groups, autoreactive groups, dyes and other aromatic compounds.

In a preferred embodiment of the invention the solid phase will cause DNA to be bound to it at one pH in preference to contaminants in the blood sample and will allow the bound nucleic acid to be released when it is contacted with an eluant at a different pH. This system can be used with a solid phase which incorporates histidine or a polyhistidine which will tend to bind nucleic acids at low pH e.g. less than 6 and will then release the bound nucleic acids when the pH is increased e.g. to greater than 8. Alternatively the nucleic acids are bound at substantially neutral pH to an aminated surface and released at very high pH.

In another embodiment of the invention a plastic moulding can incorporate a binding agent e.g. in a well in a plate etc. so that the binding agent is incorporated in the surface, the blood sample is then contacted with the surface so as to cause nucleic acids to be bound to the surface. The blood sample is then removed and the surface treated with an eluting agent to release the bound nucleic acids. When the surface is part of a well in a multi-well plate, the total system can be readily adapted for rapid large scale sampling and extraction techniques.

Binding agents which can be used include charge switchable ion exchange resins using a positively charged solid phase that can be reversed or made neutral by changing the pH above its pKa. e.g. nucleotides, polyamines, imidazole groups and other similar reagents with a suitable pKa value.

Also, nucleic acids can be bound by intercalation using a variety of intercalating compounds incorporated into the solid phase e.g. Actinomycin D, Ethidium Bromide etc.

In a further embodiment of the invention a plastic surface can be modified to include functional groups. The plastic can be any plastic used for containing samples e.g. polypropylene. The functional groups can be positively or negatively charged so as to bind the nucleic acids in the correct buffer solution.

Alternatively the functional groups can be chemical groups capable of covalent coupling to other ligands or polymers.

When the plastic is used in a plastic moulding e.g. in a well in a plate, or as a Polymerase Chain Reaction (PCR) tube, the surface characteristics of the plastic can be suitably modified for use in the present invention by including or adding the appropriate chemicals in the moulding compound e.g. as in an injection moulding compound.

When this is used in a PCR tube or in a deep well plate the tubes or wells can be used to isolate and immobilise small quantities of DNA or RNA generating a pure template for subsequent PCR or other genetic analysis and manipulation.

When the plastic is polypropylene e.g. it is in the form of a thin walled PCR tube the polypropylene surface can be modified by oxidising the surface with an oxidising agent such as potassium permanganate and sulphuric acid to create a carboxylated surface (COOH groups). This tube can then be used to improve the isolation of DNA from solutions or from crude samples e.g. blood. By adjusting the pH, di-electric constant, solubility or ionic strength the DNA or RNA can be immobilized on the walls of the tube, washed free of contaminants, ready for PCR or other analytical techniques.

The carboxy groups can be further modified by covalently coupling an anionic group such as imidazole or polyhistidine or any strong or weak ion exchanger, to allow binding of nucleic acids by a charge interaction. This tube could then be used to improve the isolation of DNA from solutions or from crude samples e.g. of blood. Again by adjusting the pH, dielectric constant, or ionic strength the DNA or RNA can be immobilised on the walls of the tube, washed free of contaminants, ready for PCR or other analytical techniques.

The nucleic acids can be eluted with in a low salt buffer so that it is ready for PCR or other analysis.

The solid phase can be contacted with a blood sample by mixing with the solid phase in a mixing/stirring device, by passing the blood sample over the solid phase or the solid phase can be paramagnetic and manipulated by a magnetic field. Although the invention is particularly suitable for the separation or isolation of nucleic acids from blood it can be used with a range of biomolecules particularly those that require removal of cell wall debris or insoluble particles.

In a preferred embodiment of the invention the solid phase is in granular form in a column and the blood sample is drawn up through the column by means of a pressure differential being applied through the column, the blood sample is drawn up with air and the granular solid material can become fluidised thus increasing the mixing and contacting rates and minimising clogging.

The method of the invention is suitable for use in a multi-well format when a series of extractions from different samples can take place substantially simultaneously and this will facilitate the automation of the extraction process allowing rapid high throughput extraction to take place and to allow combinational chemistry to be performed. This will enable there to be a high throughput in a standard well array e.g. an eight by twelve array so that a large number of sample types can be treated automatically at the same time.

The invention is described in the Example.

### EXAMPLE 1

### Extraction of Nucleic Acids from Whole Blood

A charge switchable ion-exchanger was prepared by covalently coupling polyhistidine to 100 (m glass beads using glutaldehyde by mixing 1 gram of the aminated glass beads with 0.01%(v/v) glutaldehyde in 0.1M sodium bicarbonate at pH8 containing 20 mg polyhistidine. After overnight incubation the beads were washed exhaustively to remove noncovalently bound material and stored in 10 mM MES, pH5 containing 0.1% (v/v) Tween 20.

About 300 mg of the 100(m derivitised glass beads were added to a 1 ml plastic column enclosed at both ends.

A blood sample was incubated with an equal volume of 10 mM MES pH5, containing 1% Tween 20, proteases (200(g/ml) and 1 mM EDTA. After digestion is complete the blood was sucked up the column containing the glass beads and the DNA became immobilised allowing the contaminating proteins to pass through to waste.

The glass beads containing the immobilised DNA were washed with a buffer comprising 10 mM MES pH5, containing 1% Tween 20, and 1 mM EDTA and this was repeated until the wash solution was colourless.

After washing, the beads were dried with air and DNA eluted with a small quantity of 10 mM Tris HCI, pH 8.5 and collected in a sterile tube ready for analysis. Thus the DNA were separated from the blood.

For different biomolecules, the buffer etc. can be suitably modified.

### EXAMPLE 2

One gram of carboxylated paramagnetic beads were washed in 50 mM Imidazole buffer pH6 and then mixed with 100 mg of polyhistidine in 50 ml of 50 mM Imidazole buffer pH 6. A chemical coupling agent was added (EDC) at a final concentration of 5 mg per ml and mixed overnight. The beads were washed in water, 0.5M sodium chloride, 1% Tween 20, 100 mM Tris HCl pH 8 and stored in 10 mM MES, 0.1% Tween 20 pH 5.

To extract DNA from blood, 1 mg of beads were mixed with blood diluted in 10% Tween 20 with 25 mM MES, 1 mM EDTA pH 5. The beads were separated with a magnet and washed by resuspending in 1 mM MES, 0.1% Tween 20. To elute the DNA the beads were resuspended in 10 mM Tris HCl pH 8.5 and separated with a magnet leaving the DNA in solution.

1. A method for the extraction of biomolecules from biological material which method comprises contacting the biological material with a solid phase which is able to bind the biomolecules to it at a first pH and then extracting the biomolecules bound to the solid phase by elution using an elution solvent at a second pH.

2. A method as claimed in claim 1 in which the biomolecule comprises nucleic acids.

3. A method as claimed in claim 1 or 2 in which the biological material is blood.

4. A method as claimed in claim 1, 2 or 3 in which the biomolecule is contacted with the solid phase at a pH of less than 6 to bind the DNA to the solid phase and the DNA is released from the solid phase at a pH of greater than 8.

5. A method as claimed in claim 1, 2 or 3 in which the biomolecule is contacted with the solid phase at a substantially neutral pH to bind the DNA to the solid phase and the DNA is released from the solid phase at a pH of greater than 10.

6. A method as claimed in any one of claims 3 to 5 in which the cells in the blood are lysed to release nucleic acids

7. A method as claimed in claim 6 in which the cells are lysed by contacting with an ionic or non ionic detergent, hypotonic solutions of a salt, a protease, a chaotropic agents or by use of pH changes or heat.

8. A method as claimed in any one of claims 3 to 7 in which the blood is diluted with water or other diluent in order to make it easier to manipulate and to pour.

9. A method as claimed in claim 8 in which a dilution of up to ten times is used.

10. A method as claimed in any one of the preceding claims in which the solid phase with which the biological material is contacted is formed of a material which has a natural affinity for nucleic acids

11. A method as claimed in any one of the preceding claims in which the solid phase with which the biological material is contacted is formed of a material which has its surface treated with an agent which will cause nucleic acids to bind to it or increase its affinity for nucleic acids.

12. A method as claimed in claim 11 in which the surface of the solid material is treated with a substance which can introduce a positive charge or a hydrophilic or hydrophobic surface on the solid phase

13. A method as claimed in claim 11 in which the binding agent used is a charge switchable ion exchange resins using a positively charged solid phase that can be reversed or neutralised by changing the pH above or below its pKa.

14. A method as claimed in claim 13 in which the binding agent is a nucleotide, a polyamine or a compound containing an imidazole moiety.

15. A method as claimed in claim 11 in which the nucleic acids are bound by intercalation using an intercalating compound incorporated into the solid phase.

16. A method as claimed in claim 15 in which the dye is Actinomycin D or Ethidium Bromide.

17. A method as claimed in claim 11 in which surface of the solid material is treated with histidine or a polyhistidine.

18. A method as claimed in any one of the preceding claims in which the solid phase is a controlled pore glass, a polysaccharide, a ceramic material or a porous plastic material.

19. A method as claimed in any one of the preceding claims in which the solid phase comprises beads of polystyrene or of a para magnetic material.

20. A method as claimed in any one of the preceding claims in which the solid phase comprises a plastic surface modified to include functional groups.

21. A method as claimed in claim 20 in which the plastic is polypropylene.

22. A method as claimed in claim 21 in which the polypropylene surface is modified by oxidising the surface with an oxidising agent to create a carboxylated surface.

23. A method as claimed in claim 22 in which the carboxyl groups are further modified by covalently coupling an anionic group such as imidazole or polyhistidine or any strong or weak ion exchanger, to allow binding of nucleic acids by a charge interaction.

24. A method as claimed in any one of claims 11 to 23 in which the functional groups are positively or negatively charged so as to bind the nucleic acids.

25. A method as claimed in any one of claims 11 to 23 in which the functional groups are chemical groups capable of covalent coupling to other ligands or polymers.

26. A method as claimed in any one of claims 20 to 25 in which the solid material is a porous plastic plug which is a single moulded part or is an insert in a container.

27. A method as claimed in claim 20 to 25 in which the plastic is in a plastic moulding.

28. A method as claimed in claim 27 in which the solid material is a Polymerase Chain Reaction (PCR) tube

29. A method as claimed in claim 27 in which the solid material is a deep well plate.

30. A method as claimed in any one of claims 26 to 29 in which the tubes or wells are used to isolate and immobilise small quantities of DNA or RNA generating a pure template for subsequent PCR or other genetic analysis and manipulation.

31. A method as claimed in any one of the preceding claims in which the solid phase comprises granular solid material which is contacted with a blood sample by mixing with the solid material in a mixing/ stirring device, by passing the blood sample over the solid phase or the solid phase is manipulated on a magnetisable support.

32. A method as claimed in any one of the preceding claims in which the containers are wells in a multi-well plate and a series of extractions of DNA from different samples takes place substantially simultaneously.

33. A method as claimed in any one of the preceding claims in which the solid phase is in granular form in a column and the blood sample is drawn up through the column by means of a pressure differential being applied through the column.

34. A method as claimed in claim 33 in which the blood sample is drawn up with air and the granular solid material becomes fluidised.

35. A method as claimed in any one of the preceding claims in which the biomoleclues are removed by elution with a low salt buffer solution or water.
